(19) United States
(12) Patent Application Publication
(10) Pub. No.: US 2007/0218545 A1
(43) Pub. Date: Sep. 20, 2007

(54) ANTIBODY CHIP, ANTIGEN MEASURING APPARATUS AND LIQUID DISCHARGING METHOD

(75) Inventors: Ikuo Uematsu, Kanagawa (JP); Shingo Kasai, Kanagawa (JP); Kenichi Uchiyama, Kanagawa (JP); Tomohiro Kita, Kanagawa (JP); Kayoko Oomiya, Kanagawa (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KABUSHIKI KAISHA TOSHIBA, Tokyo (JP)

(21) Appl. No.: 11/566,857

(22) Filed: Dec. 5, 2006

(30) Foreign Application Priority Data
Mar. 17, 2006 (JP) 2006-074597

(51) Int. Cl. C12M 1/34

(52) U.S. Cl. 435/287.2

(57) ABSTRACT

It is an object to provide an antibody chip and a light source measuring apparatus which are excellent in a reproducibility and a workability of a measurement and can easily be utilized. A light source measuring apparatus (1) includes, as a main structure, a concentration measuring device (2), an antibody chip (3) and a cell washing device (4). The concentration measuring device (2) serves to measure a concentration of an antigen in a specimen solution taken by the antibody chip (3) The cell washing device (4) has solution injecting means constituted by an injecting pump (44), an injecting tube (45), a first solution vessel (46), a second solution vessel (47) and a solution selecting valve (48) and solution discharging means constituted by a plurality of discharging pumps (51), a discharging tube (52), an intake end (52a) and the like. In a state in which a container cover (8) is opened, an inner part of a cell (39) of the antibody chip (3) having an antibody fixing layer formed on a main surface of a substrate (37) on a chip carrier (7) is washed to stabilize the antibody fixing layer without the solution remaining in the cell.

### BACKGROUND OF THE INVENTION

1. Field of the Invention

The present invention relates to an analysis of a living substance by an antigen-antibody reaction, and more particularly to an antibody chip for analyzing a living substance in a very small amount at a high sensitivity in a specimen solution, an antigen measuring apparatus and a liquid discharging method.

2. Related Art

Conventionally, an enzyme-linked immunosorbent assay (an ELISA method) has been known as a method of measuring a small amount of components utilizing a peculiar reaction of an antigen and an antibody. There has been proposed an antigen measuring method using a photoconductive waveguide applying the method described above. In an immune sensor to be used in the antigen measuring method, a pair of gratings is formed in light incident and emitting portions on a surface of a substrate, and a single photoconductive waveguide layer is formed on the surface of the substrate positioned between the gratings. In the structure, an antibody fixing film is formed on the photoconductive waveguide layer.

In the immune sensor having such a structure, when a specimen solution containing an antigen to be measured is caused to come in contact with the antibody fixing film, the antibody and the antigen to be measured are coupled to each other. When a fluorescent-labeled antibody is added, furthermore, an immune complex constituted by an antibody/an antigen to be measured/a fluorescent labeled antibody is formed on the surface of the substrate. In such a state, a laser beam is incident on the photoconductive waveguide layer through the grating to generate an evanescent wave and to detect an amount of a fluorescence of a specimen solution which is caused by a reaction of the evanescent wave and the immune complex through a photoreceptor, thereby analyzing a quantity of biomolecules in the specimen solution (for example, see Japanese Patent Application Laid-Open No. 8-285851).

In a conventional antigen measuring method, however, it is necessary to detect the amount of a fluorescence which is surface-emitted, and a detection sensitivity is limited. For this reason, the method is not suitable for analyzing the biomolecule in the very small amount of the specimen solution. Therefore, the inventors have disclosed a technique for improving a performance of the antigen measuring method and considerably enhancing precision (for example, see Japanese Patent Application Laid-Open No. 2005-99011) The technique has the following main points. Specifically, the evanescent wave generated by the incident light (for example, the laser beam) which is fully reflected by the photoconductive waveguide layer is partially absorbed into the immune complex in the antibody fixing film and the light incident on the antibody fixing film has an intensity attenuated and is thus reflected fully to be a reflected light. The amount of attenuation of the intensity of the reflected light has a high correlation with the amount of the immune complex. Therefore, the intensity of the reflected light is measured to measure the amount of the immune complex and that of the antigen. There have been proposed an antibody chip capable of analyzing a substance in the specimen substance in a very small amount at a high sensitivity with high precision, an antigen measuring apparatus, an antigen measuring method, and a pallet and an antibody chip packing member for easily handling the antibody chip.

In the antigen measuring method based on the technique described in Japanese Patent Application Laid-Open No. 2005-99011, a substance to be measured is a solution and a very small amount of a biological substance therein is analyzed in the practical utilization. For this reason, it is important that an immune complex generated in a cell of the antibody chip is controlled with a high reproducibility and high precision by an antigen-antibody reaction. Therefore, it is essential that cleaning is carried out without a residual solution in the cell in the antibody chip in order to stabilize a state on the antibody fixing film in the measurement of the antigen.

In the practical utilization, moreover, it is important that a measuring work thereof is simple and skills are not required.

### BRIEF SUMMARY OF THE INVENTION

In consideration of the circumstances, it is an object of the present invention to provide an antibody chip, an antigen measuring apparatus and a liquid discharging method which are excellent in a reproducibility and a workability of a measurement and can easily be practically utilized.

In order to attain the object, an antibody chip according to an embodiment of the present invention comprises: a substrate having a light transmitting property; an antibody fixing layer formed on a main surface of the substrate; a pair of optical elements for causing a light to be incident in the substrate, irradiating the light on the underneath of the antibody fixing layer and then emitting the light to an outside of the substrate; a water repellent layer formed on the main surface of the substrate in order to surround the antibody fixing layer; and a frame having one of ends which is fixed to the main surface of the substrate and serving to form a cell in order to surround the water-repellent layer, wherein a surface of the substrate surrounded by the frame is constituted by the antibody fixing layer and the water-repellent layer region.

An antigen measuring apparatus according to an embodiment of the present invention comprises an antibody chip including a substrate having a light transmitting property, an antibody fixing layer formed on one of main surfaces of the substrate, a pair of optical elements for causing a light to be incident in the substrate, irradiating the light on the underneath of the antibody fixing layer and then emitting the light to an outside of the substrate, and a frame having one of ends which is fixed to one of the main surfaces of the substrate and surrounding the antibody fixing layer to form a cell; a light emitting element for causing the light to be incident toward a pair of the optical elements which is provided on an incident side from the other main surface of the substrate; a light receiving element for detecting the light emitted from the other main surface of the substrate through the pair of the optical elements on an emitting side; and solution injecting means for injecting a predetermined solution into the cell of the antibody chip and solution discharging means for discharging the solution to an outside of the cell, wherein a discharging tube constituting the solution discharging means and serving to discharge the solution in the cell is formed to freely move into/out of the cell.

According to an embodiment of the present invention, there is provided a method of discharging a liquid from a cell of an antibody chip, the antibody chip comprising: a substrate having a light transmitting property; an antibody fixing layer formed on a main surface of the substrate; a pair of optical elements for causing a light to be incident in the substrate, irradiating the light on the underneath of the antibody fixing layer and then emitting the light to an outside of the substrate; a water-repellent layer formed on the main surface of the substrate in order to surround the antibody fixing layer; and a frame having one of ends which is fixed to the main surface of the substrate and serving to form a cell wall surface by a plane portion or a first curved portion and a second curved portion having a smaller radius of curvature than the curved portion or a corner portion in order to surround the water-repellent layer, a surface of the substrate surrounded by the frame being constituted by the antibody fixing layer and the water-repellent layer region, wherein a discharging tube is disposed in the second curved portion or the corner portion of the cell in order not to come in contact with the substrate and a pressure reducing apparatus connected to the discharging tube is driven to discharge the liquid present in the cell. In this case, if the cell takes a cylindrical shape, the discharging tube is disposed in the central part of the cell. On the other hand, in the case in which the cell takes such shape that a cell wall is formed by a plane portion or a first curved portion and a second curved portion having a smaller radius of curvature than that of the first curved portion or a corner portion, the discharging tube is disposed close to the second curved portion or the corner portion.

According to the structure of the present invention, it is possible to provide an antibody chip and a light source measuring apparatus which are excellent in a reproducibility and a workability of a measurement and can easily be practically utilized.

### BRIEF DESCRIPTION OF THE DRAWINGS

FIG. 1 is a schematic view showing a light source measuring apparatus according to an embodiment of the present invention,

FIG. 2 is a top view showing an antibody chip according to the embodiment of the present invention,

FIG. 3 is a view taken along a Y-Y line in FIG. 2,

FIGS. 4A to 4D are top views showing a cell of the antibody chip according to the embodiment of the present invention,

FIGS. 5A to 5D are top views showing another cell of the antibody chip according to the embodiment of the present invention,

FIGS. 6A to 6D are longitudinal sectional views showing a tip of a discharging tube according to the embodiment of the present invention,

FIGS. 7A to 7D are longitudinal sectional views showing a tip of another discharging tube according to the embodiment of the present invention,

FIGS. 8A to 8D are longitudinal sectional views showing a tip of a further discharging tube according to the embodiment of the present invention,

FIG. 9 is a flowchart showing a process for an antigen measuring method using an antibody chip according to the embodiment of the present invention,

FIGS. 10A to 10D are explanatory views showing the antigen measuring method using an antibody chip according to the embodiment of the present invention, and

FIGS. 11A and 11B are sectional views showing a method of injecting/discharging a solution in the cell of the antibody chip according to the embodiment of the present invention.

### DETAILED DESCRIPTION OF PREFERRED EMBODIMENTS

A preferred embodiment according to the present invention will be described below with reference to the drawings. The same or similar portions have common designations and repetitive description will not be given. However, the drawings are typical and a ratio of dimensions is different from an actual one.

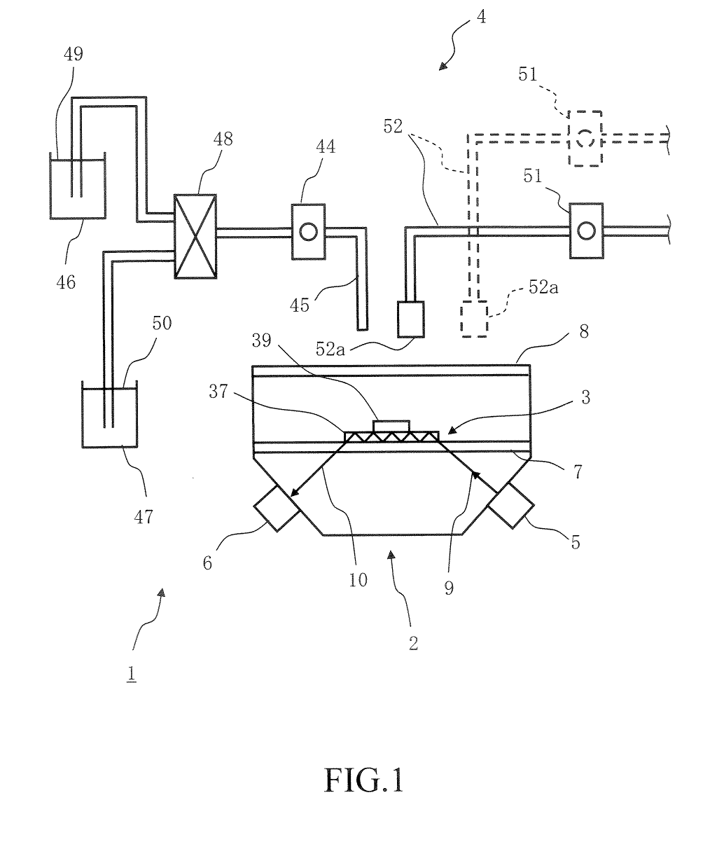

As shown in FIG. 1, a light source measuring apparatus 1 comprises, as a main structure, a concentration measuring device 2, an antibody chip 3 and a cell washing device 4. The concentration measuring device 2 serves to measure a concentration of an antigen in a specimen solution taken into the antibody chip 3. The cell washing device 4 washes the inside of a cell 39 of the antibody chip 3 having an antibody fixing layer formed on a main surface of a substrate 37 and brings a state in which a solution does not remain in the cell and the antibody fixing layer is thus stabilized. The structures of the concentration measuring device 2, the antibody chip 3 and the cell washing device 4 will be described below in detail, respectively.

### (Structure of Concentration Measuring Device)

The concentration measuring device 2 includes a light source module 5 provided on one of side surfaces and a detector 6 provided on the other side surface as shown in FIG. 1. A chip carrier 7 is mounted thereabove and a plurality of the antibody chips 3 is accommodated in the chip carrier 7. When the chip carrier 7 is mounted, a container cover 8 is closed. A laser beam is emitted from the light source module 5 and the inside of the antibody chip 3 is fully reflected as an incident light 9, and an intensity of a reflected light 10 is measured by the detector 6.

### (Antibody Chip)

The antibody chip 3 according to the embodiment comprises the substrate 37 constituting a photoconductive waveguide layer in which a light can be propagated by a full reflection and an antibody fixing layer 38 formed on at least a part of a surface for fully reflecting the photoconductive waveguide layer. It is preferable that the antibody fixing layer 38 be formed in the cell 39. The cell 39 is fixed to the surface of the substrate 37 in such a structure as to be surrounded by a frame 40 and has a water-repellent film 42 which is opened to form a reacting hole 41 in such a manner that a surface is higher than the antibody fixing layer 38 and at least a part of the antibody fixing layer 38 is exposed. The water-repellent film 42 surrounds the antibody fixing layer 38.

The frame 40 is directly bonded to a surface of a main plane constituting a full reflecting surface of the substrate 37 forming the photoconductive waveguide layer with a UV curing adhesive in order to close one of ends which is opened. The frame 40 serves to surround a measuring area in order to prevent the leakage of chemicals such as a reagent, a specimen solution and a washing solution which are put in the reacting hole 41 at an unintended time as will be described below. Accordingly, the surface of the substrate defined by an upper end of the frame 40 is formed to be higher than the water-repellent film 42.

An incident side grating 43a and a reflecting side grating 43b are formed on both sides of the cell 39 (both side in a longitudinal direction of the antibody chip 3) in the surface of the substrate 37 constituting the photoconductive waveguide layer. It is suitable that the gratings 43a and 43b be covered with the water-repellent film 42.

In the antibody chip 3, the substrate 37 is formed by a light transmitting material and a borosilicate glass is suitably used, for example.

The antibody fixing layer 38 has a structure in which an antibody is fixed by a crosslinking polymer, for example. Examples of the crosslinking polymer to be used in the antibody fixing layer 38 include a polymer containing a hydrogen bonding functional group such as photocrosslinking polyvinyl alcohol.

The antibody fixing layer 38 is constituted by protein and a material film for fixing a first antibody to cause an antigen-antibody reaction with a specimen as will be described below. The antibody is generally hydrophilic. For this reason, it is preferable that the antibody fixing layer 38 also be hydrophilic. A thickness (a distance from the surface of the photoconductive waveguide layer to that of the antibody fixing layer) is preferably 30 nm to 500 nm, is more preferably equal to or smaller than 100 nm, and is particularly preferably equal to or smaller than 80 nm.

It is preferable that the frame 40 be formed by a resin having a color such as black. Any resin material can be used as long as it has neither a reactivity nor a compatibility with a reagent, a solvent and the like but has a high moldability. It is possible to select and use an acrylic resin, an acrylonitrile-butadiene-styrene (ABS) resin and the like depending on a structure of a kit.

The water-repellent film 42 is stuck by screen printing in order to entirely cover at least a part of the antibody fixing layer 38 and portions other than the region provided with the frame 40 in the surface of the main plane constituting the whole reflecting surface of the substrate 37. The water-repellent film 42 adheres to a cell internal wall 40a of the frame 40. It is preferable that a light shielding resin having a color such as black be used for the water-repellent film 42 in order to prevent the leakage of a light incident from a lower part of the substrate 37 to an outside. If the water-repellent film 42 has neither a reactivity nor a compatibility with a kit but has a high water-repellency, there is no particular restriction. In particular, a fluorinated resin is suitable.

It is preferable that the incident side grating 43a and the reflecting side grating 43b be formed of titanium oxide (TiO₂), tin oxide (SnO₂), zinc oxide, lithium niobate, gallium arsenide (GaAs), indium tin oxide (ITO), polyimide or the like, for example. The gratings 43a and 43b have an optical function for introducing a laser beam into the antibody chip 3 and reflecting the same. If the same functions can be implemented by using other members, however, it is not particularly required. If the same functions can be implemented, moreover, another optical element such as a prism may be disposed.

In the antibody chip 3 described above, the cell 39 includes the antibody fixing layer 38 and a solution such as a reagent, a specimen solution or a washing solution is injected into the reacting hole 41 in an inner part as will be described below. Moreover, the solution is discharged from the cell 39. As described above, in order to wash the inner part of the cell 39 in the measurement of the antigen and to stabilize the state of the antibody fixing layer 38, it is important that the solution is prevented from remaining in the cell 39 in the process for washing the inner part of the cell 39. It is desirable that the work for the washing treatment can easily be carried out without requiring the skills.

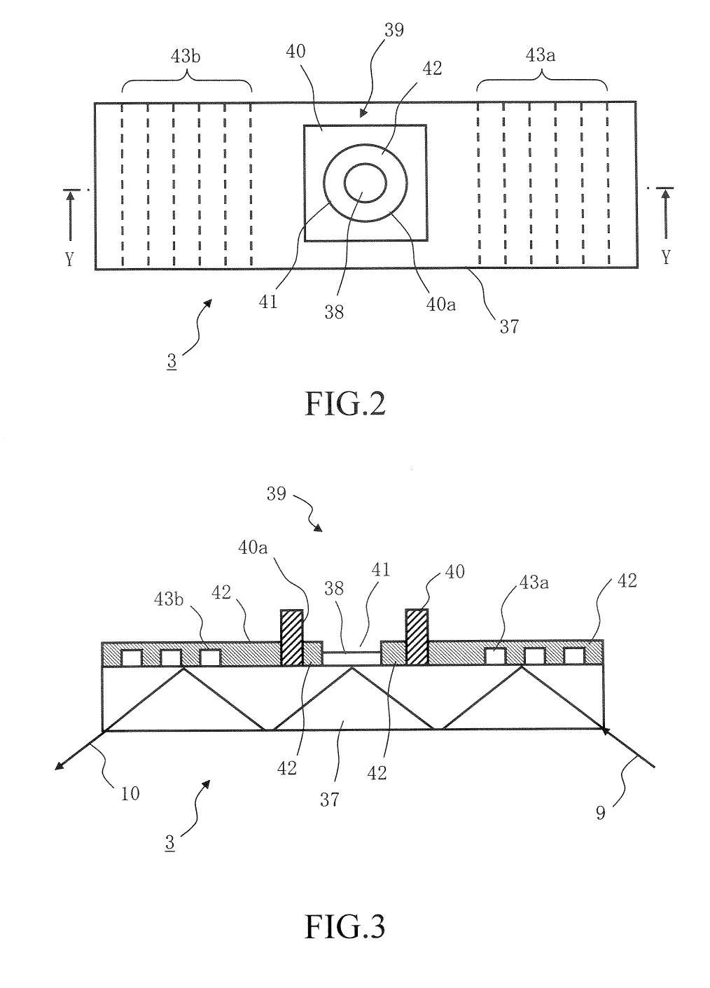

For this purpose, portions other than the hydrophilic antibody fixing layer 38 in the cell 39 are constituted by a water-repellent material as described above, and furthermore, the shape of the inner part is important. Regarding the suitable standard shape, the shapes of the upper surfaces of the antibody fixing layer 38 and the cell internal wall 40a of the frame 40 are concentric with each other and the upper surface of the water-repellent film 42 takes the shape of a donut as shown in FIGS. 2 and 3.

By setting the inner part of the cell to have such a shape and arrangement, a frequency at which the solution remains in the cell is comparatively low. Even if the solution remains, moreover, there is a tendency that the solution is gathered in a comparatively central region. Therefore, an intake end for discharging the solution in the washing process is provided in a central region of the cell 39. Thus, it is possible to efficiently eliminate the solution.

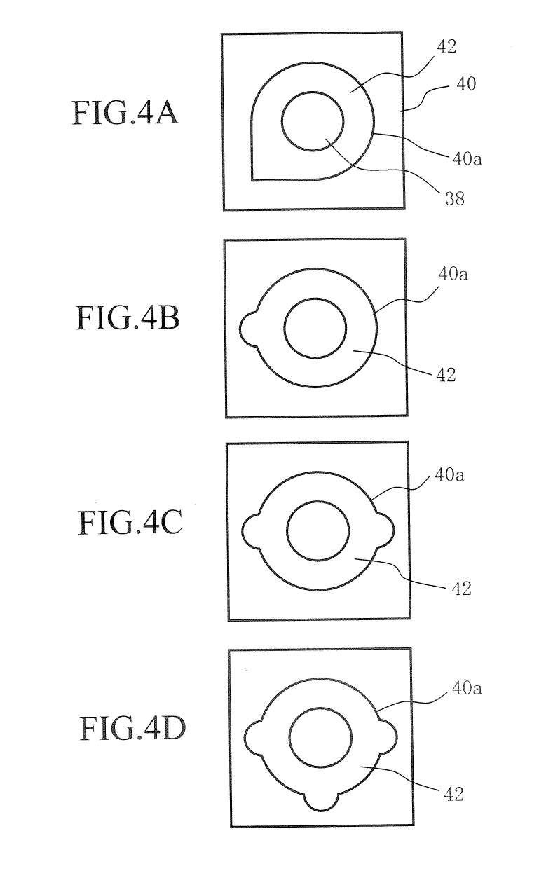

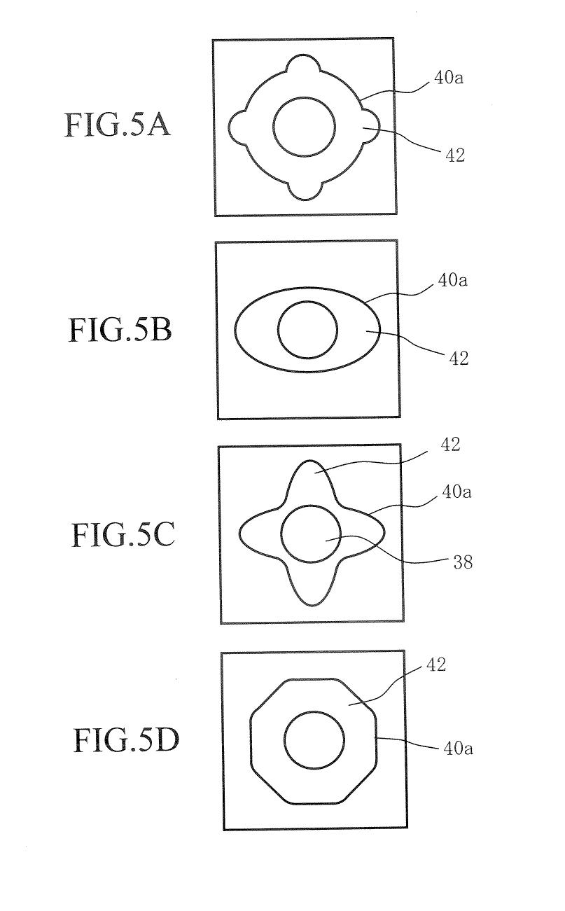

While the sectional shape of the cell 39 is circular in the embodiment, other various shapes can be taken. A variant will be described with reference to FIGS. 4 and 5. FIGS. 4 and 5 are top views showing the cell 39.

In FIG. 4A, the circular antibody fixing layer 38 is formed in the same manner as in FIG. 2 and the water-repellent film 42 is bonded to the substrate 37 in close contact with the cell internal wall 40a in order to surround the antibody fixing layer 38 except that one corner portion is formed on the cell internal wall 40a.

In FIG. 4B, an arcuate retreat portion is formed in one portion of the cell internal wall 40a, that is, a portion is formed by recessing the cell internal wall like a circular arc.

In FIG. 4C, the arcuate retreat portion is formed in two portions of the cell internal wall 40a.

In FIG. 4D, the arcuate retreat portion is formed in three portions of the cell internal wall 40a.

In FIG. 5A, the arcuate retreat portion is formed in four portions of the cell internal wall 40a.

In FIG. 5B, the cell internal wall 40a is formed elliptically.

In FIG. 5C, a retreat portion taking a similar shape to a parabolic curve is formed from a circular region in which the cell internal wall 40a is provided around the antibody fixing layer 38.

In FIG. 5D, furthermore, the cell internal wall 40a takes a shape obtained by a combination of a plane portion and a curved portion or a corner portion.

In these cells, the sensing area 38, the water-repellent film 42 and the cell internal wall 40a having a higher hydrophilicity than the water-repellent film 42 are present. The solution remaining in the cell is collected in such a form that a contact area with a hydrophobic film is the smallest in a balance of a gravity applied to the solution and a hydrophilicity with a material on a surface in the cell. In the case in which the cell internal wall has such a structure that a plane portion or a curved portion and a curved portion having a smaller radius of curvature than the curved portion or a corner portion are close to each other in the cell internal wall, the solution tends to gather at the curved portion having a small radius of curvature or the corner portion. Accordingly, it is possible to efficiently carry out a sucking work by causing a tip of a sucking pipette to be close to the vicinity of the curved portion having a small radius of curvature or the corner portion, thereby sucking the solution.

### (Cell Washing Device)

The cell washing device 4 comprises an injecting tube 45 connected to an injecting pump 44 as solution injecting means for injecting a solution into the cell 39 of the antibody chip 3 as shown in FIG. 1. A syringe pump or the like is suitable for the injecting pump 44. In order to select the solution to be injected, moreover, there is provided a solution selecting valve 48 connected between a first solution vessel 46 or a second solution vessel 47 and the injecting pump 44. For example, a washing solution 49 containing a surfactant is put in the first solution vessel 46 and a buffer solution 50 is put in the second solution vessel 47. Depending on the necessity of a measuring procedure, it is possible to automatically select either of the solutions which is to be injected to the reacting hole 41 of the cell 39.

It is preferable that a lower end to be an injecting port of the injecting tube 45 be positioned in the vicinity of a part provided just above the antibody fixing layer 38 in the cell 39. Thus, the solution to be injected can be reliably dropped onto the antibody fixing layer 38.

As solution discharging means for discharging a solution from the inner part of the cell 39, a discharging tube 52 connected to a discharging pump 51 is provided as shown in FIG. 1. It is preferable that a bimorph pump or the like be used for the discharging pump 51. An amount of discharge of the discharging pump 51 is suitably 12 L (liters)/min. A plurality of the discharging pumps 51 or the discharging tubes 52 may be provided as shown in a dotted line of FIG. 1.

It is preferable that the tip of the discharging tube 52, for example, the intake end 52a such as a pipette be positioned in the vicinity of the cell internal wall 40a of the frame 40. Thus, the solution overflowing from the reacting hole 41 flows toward the cell internal wall 40a side of the frame 40 and is reliably discharged from the discharging tube 52 provided on the cell internal wall 40a because the water-repellent film 42 has a water repellency.

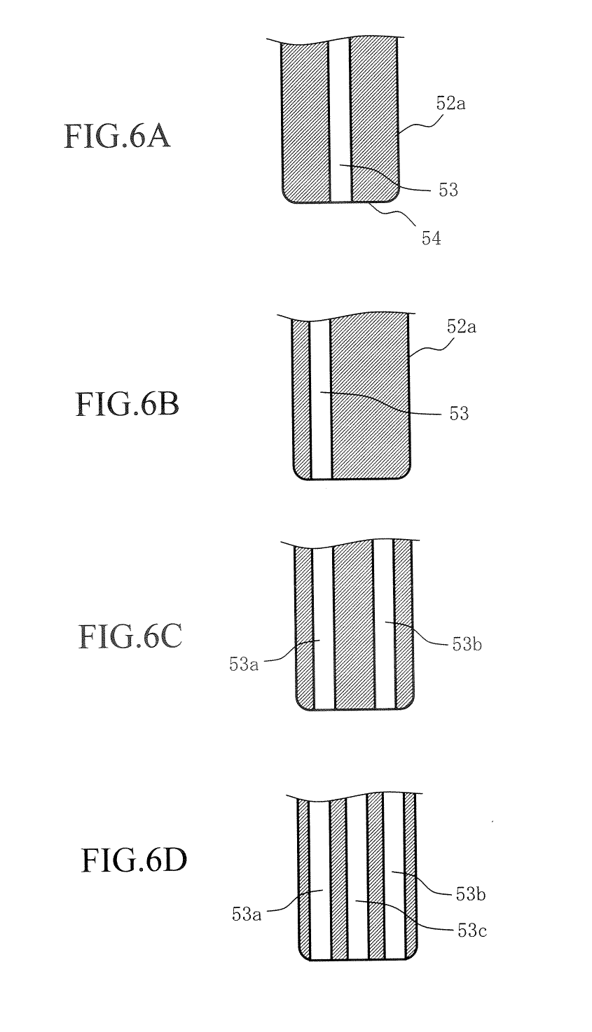

As described above, it is very important that the solution such as a washing solution is prevented from remaining in the cell 39 in order to maintain a high reproducibility and workability in the measurement of a very small amount of living substances. Therefore, the shape of the intake end 52a of the discharging tube 52 is important. The preferred aspect will be described with reference to FIGS. 6 to 8. FIGS. 6 to 8 are longitudinal sectional views showing the intake end 52a.

FIGS. 6A to 6D show a standard structure of the intake end 52a. The intake end 52a takes a cylindrical shape having a circular cross section, and a tip surface thereof has a planar flat surface 54. In FIG. 6A, an intake hole 53 is formed along a central axis of the intake end 52a. In FIG. 6B, the intake hole 53 is formed eccentrically toward an outside diameter side of the intake end 52a. In FIG. 6C, two intake holes 53a and 53b are formed. In FIG. 6D, three intake holes 53a, 53b and 53c are formed.

The outside diameter of the intake end 52a is smaller than an inside diameter of the cell internal wall 40a of the frame 40 shown in FIG. 2. It is suitable that a dimensional difference between the outside diameter of the intake end 52a and the inside diameter of the cell internal wall 40a be 0.02 mm to 10 mm. The outside diameter of the intake end 52a is set to be greater than the bore of the reacting hole 41. Consequently, the solution in the cell 39 can be discharged very easily in the washing work so that the workability can be enhanced. In the discharging work using the discharging tube 52, moreover, the intake end 52a does not come in sliding contact with the antibody fixing layer 38 so that the mechanical damage of the antibody fixing layer 38 is not generated at all.

In the discharging work using the discharging tube 52 having the intake end 52a, furthermore, the flat surface 54 of the intake end 52a is preferably disposed in parallel with the main plane of the substrate 37 in such a manner that a distance from the surface of the antibody fixing layer 38 is 0.01 mm to 5 mm. By the disposition in such a distance, the solution in the cell 39 can be discharged completely so that the inner part of the cell 39 becomes clean and the surface of the antibody fixing layer 38 is stabilized. When the discharging tube 52 comes in contact with the surface of the cell 39, moreover, the cell 39 is moved. As a result, precision in the measurement is reduced. For this reason, it is preferable that the tip of the discharging tube 52 be close so as not to come in contact with the cell 39. In consideration of precision in a position control of the measuring apparatus, it is preferable that the distance have a lower limit of approximately 0.01 mm.

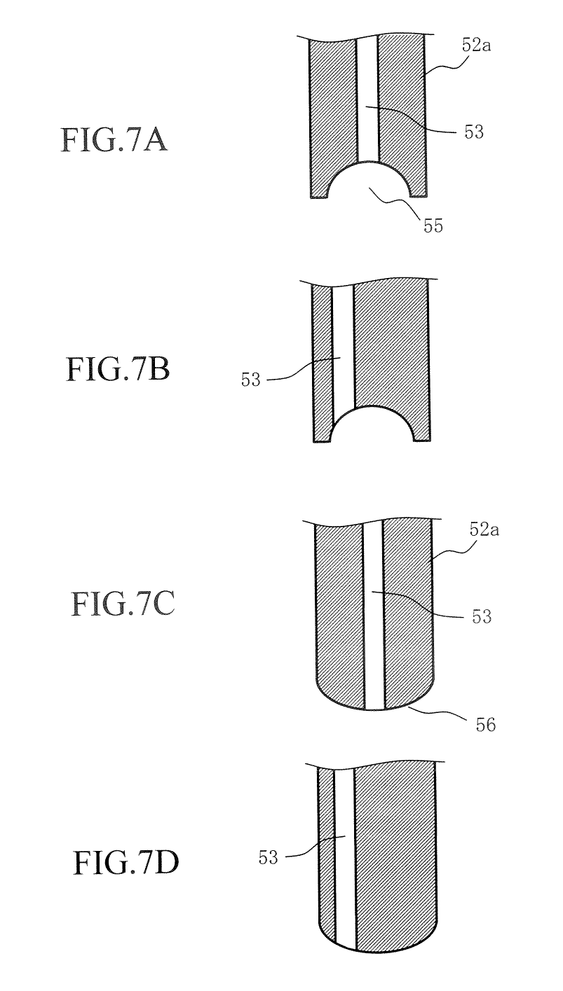

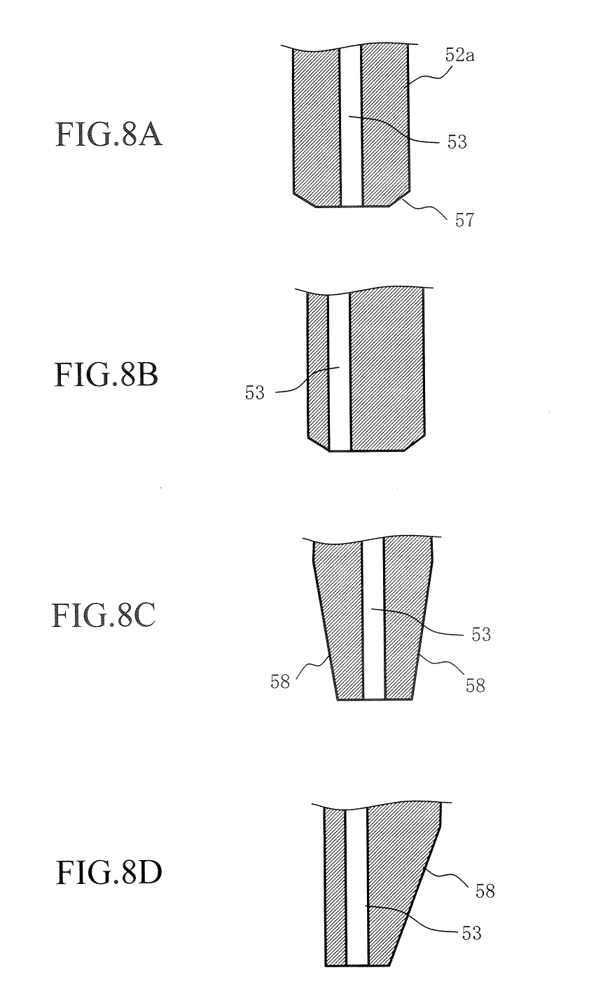

The intake end 52a can take various shapes. In FIGS. 7A and 7B, a lower end of the intake end 52a has such a structure that a concave portion 55 is formed semicircularly, for example. Depending on the circumstances, moreover, the lower end of the intake end 52a has such a structure as to include a convex portion 56 formed to be slightly convex as shown in FIGS. 7C and 7D. In this case, furthermore, an outer peripheral portion of the lower end of the intake end 52a is chamfered and has a notch portion 57 as shown in FIGS. 8A and 8B. As shown in FIGS. 8C and 8D, moreover, the lower end is tapered to have a taper portion 58.

It is suitable that the cross section of the intake end 52a is analogous to the opening shape of the cell internal wall 40a of the frame 40 in the cell 39 shown in FIGS. 4 and 5.

### (Antigen Measuring Method)

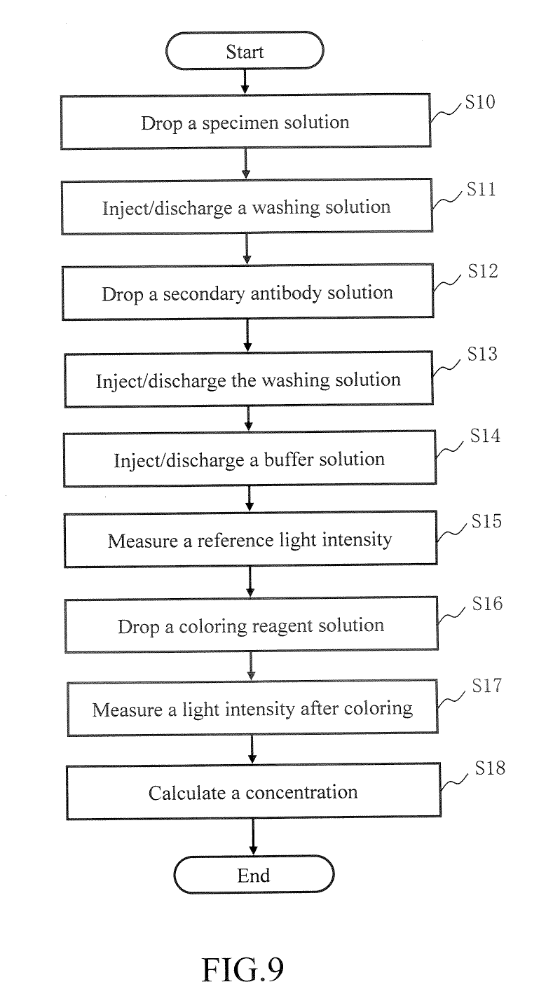

With reference to FIGS. 9, 10 and 11, next, description will be given to a method of measuring an antigen of a specific substance in a specimen solution using the antibody chip 3 according to an embodiment of the present invention.

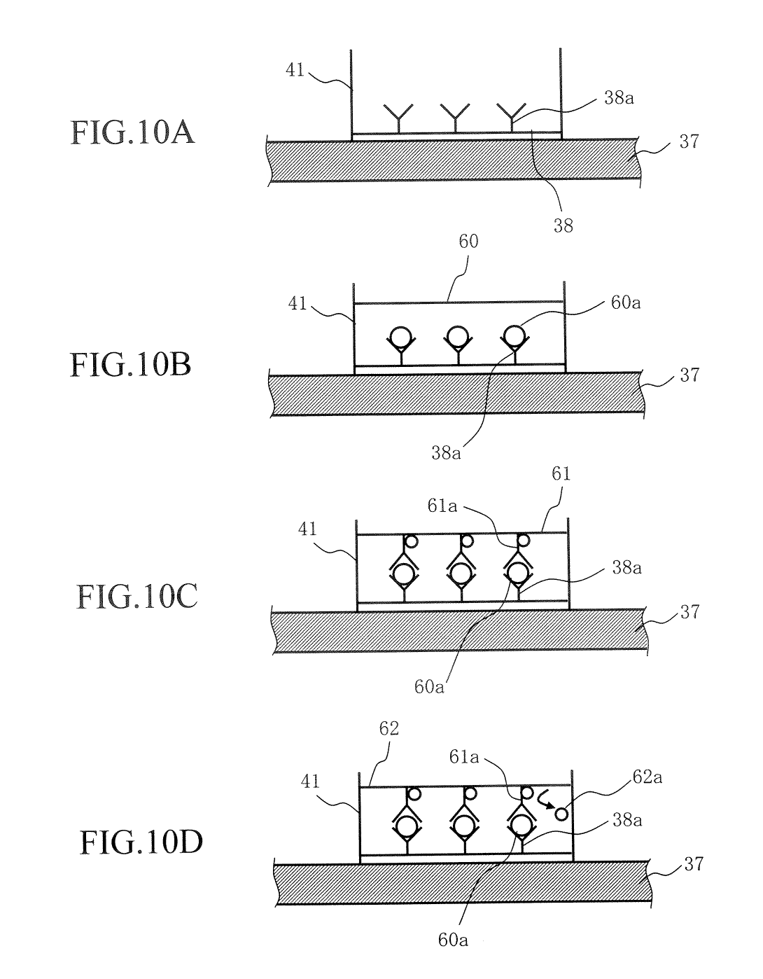

Step S10 in FIG. 9: The antibody fixing layer 38 constituted by a primary antibody 38a for peculiarly recognizing an antigen 60a to be a measuring object such as protein or a gene is formed on the surface of the substrate 37 in the bottom portion of the reacting hole 41 in the antibody chip 3 as shown in FIG. 10A. When a specimen solution 60 containing the antigen 60a is dropped onto the antibody fixing layer 38 in the reacting hole 41, the antigen 60a is bonded to the primary antibody 38a to form a primary antibody-antigen complex as shown in FIG. 10B.

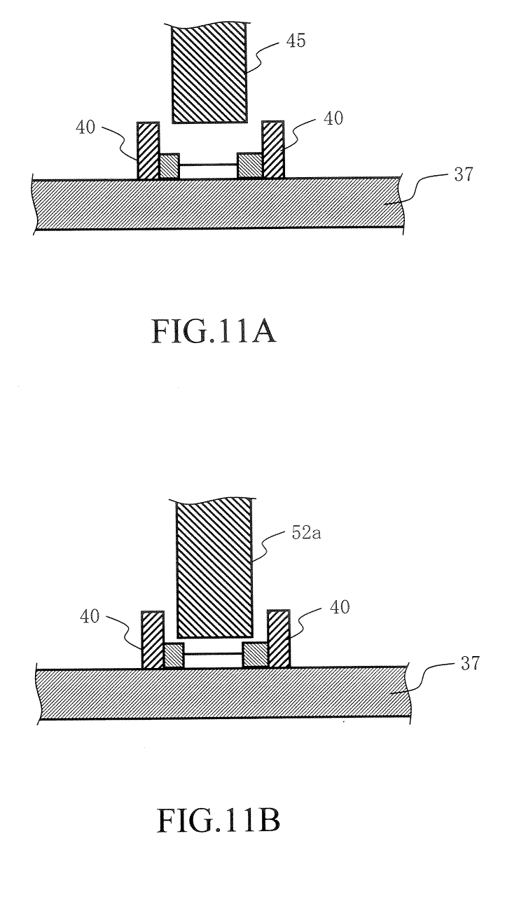

Step S11: Next, the specimen solution 60 other than the antigen 60a bonded to the primary antibody 38a is washed with a washing solution such as buffer phosphate (PBS) containing a surfactant. At the washing step, the container cover 8 shown in FIG. 1 is opened and the injecting tube 45 is disposed in the frame 40 of the antibody chip 3 as shown in FIG. 11A and a washing solution is injected in such a predetermined amount as not to overflow from the cell 39 by using the cell washing device 4. After the injecting tube 45 is separated from the frame 40, subsequently, the sucking end 52a of the discharging tube 52 is inserted into the frame 40 and the washing solution is sucked and discharged as shown in FIG. 11B. The sucking end 52a having the structure shown in FIGS. 6 to 8 is properly used corresponding to the structure of the cell 39 shown in FIGS. 2 to 5. Thus, a suitable washing operation can be caused to be executed in a simple work.

At the washing step, it is preferred that the movement and arrangement of the injecting tube 45 and the discharging tube 52 in the cell washing device 4 be carried out automatically.

At the washing step, it is very important to suck and discharge the washing solution in such a manner that the antigen 60a bonded to the primary antibody 38a to be an immune complex formed on the antibody fixing layer 38 is not disturbed. Therefore, the washing method described for the cell washing device 4 is employed to clean the inner part of the cell 39 without a residual solution, thereby stabilizing the antibody fixing layer 38.

Step S12: Next, a secondary antibody solution 61 which is enzyme-labeled is dropped. As shown in FIG. 10C, consequently, the secondary antibody 61a is further bonded to the antigen 60a in a separate portion from the primary antibody 38a. As a result, a primary antibody-antigen-secondary antibody complex is formed. For a marker enzyme labeling the secondary antibody 61a, it is possible, for example, to use peroxidase (POD) as an oxidation-reduction enzyme.

Step S13: Next, the secondary antibody solution 61 containing the secondary antibody 61a which does not form the complex is washed with the washing solution such as PBS containing a surfactant again. Also in the washing step, the same washing method as that described in the Step S11 is employed.

Step S14: Then, the surfactant used for the washing work is removed and only the PBS is injected as a buffer solution into the cell 39 in order to obtain a stabilization. Thereafter, the buffer solution is discharged from the cell 39. It is sufficient that the injection/discharge of the buffer solution is carried out by the entirely same method as that described in the Step S11.

In the washing process of the Step S13 and the process for injecting/discharging the buffer solution, it is possible to suck and discharge a solution such as a washing solution in such a manner that the primary antibody-antigen-secondary antibody complex to be the immune complex formed on the antibody fixing layer 38 is not disturbed. The inner part of the cell 39 can be cleaned without a residual solution so that the antibody fixing layer 38 can be stabilized.

Step S15: At this time, a laser beam or the like is irradiated from a semiconductor laser 25 toward the incident side grating 43a of the antibody chip 3 and a light emitted from the emitting side grating 43b is received by the photoreceptor, and a reference light intensity is thus measured.

Step S16: As shown in FIG. 10D, next, a color reagent solution 62 is dropped into the cell 39. For the color reagent solution 62, it is preferable to use 1 liter of a buffer solution having pH=4.9 containing 80 millimoles of acetic acid, 1.13 millimoles of tetramethylbenzidine (TMBZ), 1.91 millimoles of hydrogen peroxide (H₂O₂), and dimethyl sulfoxide (DMSO) of less than 1% be used, for example. Consequently, a radical oxygen atom (O*) is generated by an oxidation-reduction enzyme reaction of a labeled enzyme such as POD and H₂O₂ to be a substrate of the labeled enzyme. An enzyme reactive product 62a obtained by coloring the color reagent is generated by the O* prepared through the enzyme reaction.

Step S17: In such a state, the laser beam or the like is irradiated from the semiconductor laser 25 toward the incident side grating 43a of the antibody chip and a light reflected from the emitting side grating 43b is received by the photoreceptor to measure a light intensity after the coloring.

Step S18: A concentration of the antigen 60a to be a measuring object in the specimen solution 60 is calculated from a difference between the reference light intensity measured at the Step S15 and the light intensity after coloring which is measured at the Step S17.

When the light intensity is continuously measured by using the measuring method, it is possible to calculate the concentration by employing a value at a predetermined time from the Step S14 to the Step S15 as the reference light intensity and a value at a predetermined time after the Step S16 as the light intensity after coloring. Accordingly, it is not necessary to carry out an object experiment or the like using a non-antigen solution, thereby measuring the reference light intensity.

The antibody chip 3 according to the present invention causes the antigen-antibody reaction and the coloring reaction in the reacting hole 41. Therefore, approximately 1 microliter of the specimen solution 60 can be measured sufficiently.

In the present embodiment, the antibody chip 3 having the structure described above is used and the inner part of the cell of the antibody chip 3 is washed by using the cell washing device 4. Consequently, it is possible to stabilize the antibody stabilizing layer without mechanically damaging the antibody fixing layer. By cleaning the inner part of the cell in the antibody chip without the residual solution, it is possible to control the immune complex generated in the cell of the antibody chip with a high reproducibility and high precision by the antigen-antibody reaction. Thus, it is possible to analyze a living substance in the specimen solution in a very small amount with a high sensitivity, high precision and high reproducibility.

Moreover, the concentration of the antigen can be measured very easily by the concentration measuring device 2. In addition, the washing work including the removal of the solution in the cell of the antibody chip 3 can be carried out easily. Thus, it is possible to cause the work for measuring an antigen to be changed into a routine work without requiring skills. Thus, the measurement of the antigen according to the present invention is excellent in the reproducibility and workability of the measurement so that a practical utilization thereof can be carried out very readily.

In the measurement using the antigen-antibody reaction, the concentration of the specimen is measured depending on an increase or decrease in a reference light in place of a fluorescence. Therefore, in the case in which it is difficult to obtain a large amount of the specimen, for example, the case of an inspection for a newborn baby and a small animal or the like, the measurement according to the present invention can be utilized.

While the preferred embodiment of the present invention has been described above, the embodiment is not restricted to the present invention. Those skilled in the art can make various changes and modifications to specific embodiments without departing from the technical thought and technical range of the present invention.

For example, the washing work in the cell 39 of the antibody chip 3 using the cell washing device 4 or the work for injecting/discharging a solution may be carried out for a plurality of antibody chips 3 mounted on the chip carrier 7 on the outside of the concentration measuring device 2 differently from the case described in the present embodiment.

Moreover, a member for mounting the antibody chip 3 is not restricted to the chip carrier 7 but a plurality of antibody chips 3 may be mounted on the pallet described in Japanese Patent Application Laid-Open No. 2005-99011 by the inventors to carry out the measurement of the antigen, for example.

What is claimed is:

1. An antibody chip comprising:
a substrate having a light transmitting property;
an antibody fixing layer formed on a main surface of the substrate;
a pair of optical elements for causing a light to be incident in the substrate, irradiating the light on the underneath of the antibody fixing layer and then emitting the light to an outside of the substrate;
a water-repellent layer formed on the main surface of the substrate in order to surround the antibody fixing layer; and
a frame having one of ends which is fixed to the main surface of the substrate and serving to form a cell in order to surround the water-repellent layer,
wherein a surface of the substrate surrounded by the frame is constituted by the antibody fixing layer and the water-repellent layer region.

2. An antibody chip comprising:
a substrate having a light transmitting property;
an antibody fixing layer formed on a main surface of the substrate;
a pair of optical elements for causing a light to be incident in the substrate, irradiating the light on the underneath of the antibody fixing layer and then emitting the light to an outside of the substrate;
a water-repellent layer formed on the main surface of the substrate in order to surround the antibody fixing layer; and
a frame having one of ends which is fixed to the main surface of the substrate and serving to form a cell in order to surround the water-repellent layer, an internal wall of the cell formed by the frame being constituted by a cylindrical portion and a corner portion or a curved portion having a smaller radius of curvature than a radius of curvature of the cylindrical portion,
wherein a surface of the substrate surrounded by the frame is constituted by the antibody fixing layer and the water-repellent layer region.

3. An antibody chip comprising:
a substrate having a light transmitting property;
an antibody fixing layer formed on a main surface of the substrate;
a pair of optical elements for causing a light to be incident in the substrate, irradiating the light on the underneath of the antibody fixing layer and then emitting the light to an outside of the substrate;
a water-repellent layer formed on the main surface of the substrate in order to surround the antibody fixing layer; and
a frame having one of ends which is fixed to the main surface of the substrate and serving to form a cell in order to surround the water-repellent layer, an internal wall of the cell formed by the frame being constituted by a plane portion and a curved portion,
wherein a surface of the substrate surrounded by the frame is constituted by the antibody fixing layer and the water-repellent layer region.

4. The antibody chip according to claim 1, wherein the frame is constituted by a light shielding acrylonitrile-butadiene-styrene resin or an acrylic resin.

5. The antibody chip according to claim 1, wherein the water-repellent layer is constituted by a fluorinated resin material having a light shielding property.

6. An antigen measuring apparatus comprising an antibody chip including a substrate having a light transmitting property, an antibody fixing layer formed on one of main surfaces of the substrate, a pair of optical elements for causing a light to be incident in the substrate, irradiating the light on the underneath of the antibody fixing layer and then emitting the light to an outside of the substrate, and a frame having one of ends which is fixed to one of the main surfaces of the substrate and surrounding the antibody fixing layer to form a cell;
a light emitting element for causing the light to be incident toward a pair of the optical elements which is provided on an incident side from the other main surface of the substrate;
a light receiving element for detecting the light emitted from the other main surface of the substrate through the pair of the optical elements on an emitting side; and
solution injecting means for injecting a predetermined solution into the cell of the antibody chip and solution discharging means for discharging the solution to an outside of the cell,
wherein a discharging tube constituting the solution discharging means and serving to discharge the solution in the cell is formed to freely move into/out of the cell.

7. The antigen measuring apparatus according to claim 6, wherein a tip surface of the discharging tube is planar and is disposed in parallel with one of the main surfaces of the substrate when it is caused to enter the cell and is fixed.

8. The antigen measuring apparatus according to claim 6, wherein a tip of the discharging tube is cylindrical and has a smaller outside diameter than a diameter of an internal wall of the cell in a range of 0.02 mm to 10 mm.

9. A method of discharging a liquid from a cell of an antibody chip, the antibody chip comprising:
a substrate having a light transmitting property;
an antibody fixing layer formed on a main surface of the substrate;
a pair of optical elements for causing a light to be incident in the substrate, irradiating the light on the underneath of the antibody fixing layer and then emitting the light to an outside of the substrate;
a water-repellent layer formed on the main surface of the substrate in order to surround the antibody fixing layer; and
a frame having one of ends which is fixed to the main surface of the substrate and serving to form the cylindrical cell in order to surround the water-repellent layer,
a surface of the substrate surrounded by the frame being constituted by the antibody fixing layer and the water-repellent layer region,
wherein a discharging tube is disposed in a central part of the cylindrical cell in order not to come in contact with the substrate and a pressure reducing apparatus connected to the discharging tube is driven to discharge the liquid present in the cell.

10. A method of discharging a liquid from a cell of an antibody chip, the antibody chip comprising:
a substrate having a light transmitting property;
an antibody fixing layer formed on a main surface of the substrate;
a pair of optical elements for causing a light to be incident in the substrate, irradiating the light on the underneath of the antibody fixing layer and then emitting the light to an outside of the substrate;
a water-repellent layer formed on the main surface of the substrate in order to surround the antibody fixing layer; and
a frame having one of ends which is fixed to the main surface of the substrate and serving to form a cell wall surface by a plane portion or a first curved portion and a second curved portion having a smaller radius of curvature than the curved portion or a corner portion in order to surround the water-repellent layer,
a surface of the substrate surrounded by the frame being constituted by the antibody fixing layer and the water-repellent layer region,
wherein a discharging tube is disposed in the second curved portion or the corner portion of the cell in order not to come in contact with the substrate and a pressure reducing apparatus connected to the discharging tube is driven to discharge the liquid present in the cell.
